(19) United States
(12) Patent Application Publication
(10) Pub. No.: US 2007/0060994 A1
(43) Pub. Date: Mar. 15, 2007

(54) Blood flow diverters for the treatment of intracranial aneurysms

(76) Inventors: Riad H. Gobran, New Hope, PA (US); Ajay K. Wakhloo, Key Biscayne, FL (US); Yusuf M. Khan, Charlottesville, VA (US)

Correspondence Address: John S. Munday, Esquire, PO Box 423, Isanti, MN, 55040, US

(21) Appl. No.: 11/225,285

(22) Filed: Sep. 12, 2005

(51) Int. Cl. A61F 2/06

(52) U.S. Cl. 623/1.4; 623/1.53; 623/1.46

(57) ABSTRACT

A blood flow diverter device for treatment of intracranial aneurysms, including a porous tubular member having a central portion and two ends. The member is of sufficient flexibility and body compatibility to be placed in proximity within an intracranial aneurysm. The central portion of the tubular member has a sufficiently decreased porosity to block blood flow from entering through the aneurysm. This is done by one of three methods: (1) the central portion of the member can be compressed to decrease porosity and heat set to hold the compression; (2) the angle of the fibers can be altered if the tubular member is made from a braided fibers; or (3) a monomeric coating can be formed on the central portion in an amount sufficient to decrease the porosity of the central portion upon polymerization of the monomeric coating. In the third embodiment a polymerization initiator is provided for polymerizing the monomeric coating upon command to cause the decreased porosity to block the blood flow. The device is heat set after compression to permit insertion and expansion in the patient. The tubular member has sufficient porosity at the two ends to keep open small perforator arteries proximate to the intracranial aneurysm.

### FIELD OF THE INVENTION

This invention relates to medical devices. More particularly, the invention relates to flexible and elastic devices produced using, for example, heat-settable polymer filaments or biocompatible metals, that can be used as blood flow diverters for intracranial aneurysms that change their porosity once placed within the artery.

### BACKGROUND OF THE INVENTION

A wide variety of medical devices are now available for the treatment of intracranial aneurysms. The standard surgical approach entails after craniotomy, the placement of a clip across the neck of an aneurysm to exclude it from the main circulation. The goal is to prevent a (re)bleed into the brain from an aneurysm rupture. The standard surgical approach is being replaced by minimally endovascular techniques. Most of the techniques involve the placement of platinum coils, which are after securing them within the aneurysm pouch, detach either mechanically (Cordis, Microvention) or using electrically detachable systems (MTI, Targert/BSC). The idea of this intrasaccular approach to aneurysm treatment is to create, after coiling the sac, a stable thrombus and subsequently scarring. This prevents (re)rupture which can be fatal (ISAT, Lancet 2002, Wiebers ISUIA Lancet 2003). The placement of coils however, has shown to have some intrinsic limitations, one of the most important being the risk of aneurysm re-canalization within the first year due to coil compaction or to large size of aneurysm neck (Murayama, Journal of Neurosurgery 2003). The second most common risk is rupture of the aneurysm during placement of coils within the extreme thin-walled aneurysm sac. In general, this leads to severe neurological deficit or even death of a patient. Multiple flow studies over the past decade have shown that redirecting blood flow within the parent artery away from the aneurysm, may lead to a safe and permanent thromboocclusion of the aneurysm without the risk of damaging the aneurysm wall (Aneis J Biomech. 1999, Stancampiano Annals of Biomed. 2000, Wakhloo AJNR 1995, 1996, Lanzino, Mericle, J Neurosurgery, 1999). Tubular structures have been developed to address these issue. However, most of the current devices following that concept (Neuroform Target/BSC; Enterprise Cordis J&J) do work only in conjunction with coils because of their high porosity (ratio material free area to area covered by material). The high porosity devices alone do not uncouple the blood flow sufficiently to create a stable clot within the aneurysm sac (Lieber Annals of Biomed. 2002). A further reduction of the porosity is required however, without the risk of closing important smaller side branches, which arise in the proximity of the aneurysm and supply vital brain tissue. These so-called perforators are anywhere from 150 micron up to 1.5 mm in diameter.

In the intracranial circulation, unlike in any other parts of the body, the vascular structures are extremely tortuous. The intracranial arteries (pial vessels) are only surrounded by a watery substance, which is the cerebrospinal fluid (CSF). There is no significant surrounding soft tissue which may support the arteries. The arterial wall is extremely thin and can not easily be straightened. Straightening of the artery can lead to kinks with subsequent flow reduction and thrombosis or direct arterial tear (dissection). Flow diverters designed for the intracranial arteries have to be supple and easily adaptable to the tortuosity of the vessel boundaries. Yet, these medical devices must be strong, flexible, elastic, biocompatible, porous if they cover segments of vessel which give rise to perforators, radio-opaque, and sized correctly. Vascular blood flow diverters used in treating aneurysms in blood vessels, must be flexible enough to bend and posses a significant radial force to conform to the shape of the blood vessels into which they are inserted.

None of the prior art addresses the need to change the porosity of a device when it is used as a blood flow diverters for aneurysms. As is well known, an aneurysm occurs when the artery wall becomes thinner and bulges, forming a sack in which blood circulates. The danger is when this sack (or any aneurysm) breaks, which can lead to death. The present method of treating aneurysms involves the inserting of a coil inside the aneurysm sack to hold it up from the inside. A blood flow diverters for aneurysms would be of great advantage in the art if it could be placed at the point of leakage and in some manner reduce the porosity of the aneurysm sack at that point. Redirecting the flow of blood at an aneurysm site using a blood flow diverter is a difficult task especially in the case of cerebral aneurysms. The difficulty arises because it is essential to block blood from entering through the neck of the aneurysm while keeping open the smaller perforator arteries that are in the vicinity of the aneurysm site.

It would be of great advantage in the art if such a blood flow diverters could be developed.

Another advantage would be to provide a device which can be inserted to the desired location of an aneurysm and, in some manner, have the device decrease its porosity at the option of the medical team.

Other objects will appear hereinafter.

### SUMMARY OF THE INVENTION

It has now been discovered that the above and other advantages of the present invention may be achieved in the following manner. The invention comprises the use of a blood flow diverter formed from a porous tubular member having a central portion and two ends. The member is of sufficient flexibility and body compatibility to be placed in proximity with an aneurysm on the outside to keep blood out of the aneurysm. The central portion has reduced porosity in two separate forms to block blood flow from entering through the aneurysm.

The braided fibers are compressed to decrease the porosity of said central portion and heat set to preserve that decrease. The braided fibers are also given further decreased porosity by coating the central portion with a monomeric coating in an amount sufficient to decrease the porosity of said central portion upon polymerization of the monomeric coating. A polymerization initiator is used for polymerizing the monomeric coating upon command to cause said decreased porosity to block said blood flow after said device has been placed in proximity with an aneurysm.

In one aspect of this invention, the blood flow diverter is formed from a tubular braided polymeric flow diverter using filaments of sufficiently low diameter to prevent blockage of existing perforator arteries. When the braided polymeric flow diverter is in place, it expands to decrease the porosity of the diverter and prevent damaging leakage at the aneurysm. The porosity can be decreased in the central portion by compressing the central portion or by braiding the fibers of the central portion at an angle selected to decrease that portion's porosity. An increase of the aneurysm pressure, which potentially would lead to aneurysm rupture, cannot be expected after placement of such a device in front of the aneurysm neck.

This approach set forth above is to design a tubular braided polymeric flow diverter using filaments of sufficiently low diameter to prevent blockage of existing perforator arteries. The flow diverter will have varying porosities along its length, namely, a very low porosity (high coverage) in the part of the flow diverter that will be placed across from the neck of the aneurysm, but high porosity (low coverage) on both sides of the aneurysm. This is achieved by varying the braiding angle, during the braiding process, as well as by compressing the finished flow diverter in the longitudinal direction in such a way that the middle part is compressed much tighter than the rest, and thus obtain higher coverage, and then heat setting the device in such configuration. The technology of heat setting to achieve a specific configuration as well as the choice of suitable polymers is described in other parts of this application.

Another unique aspect of this invention is the construction of a tubular structure made from such braided materials or, alternatively, from metal, on which the surface of the structure has a monomeric material that can be polymerized at any time and particularly after the structure has been placed in proximity to an aneurysm.

This second approach to achieve the same ultimate goal is to coat all or the middle part of the flow diverter with a reactive monomer or mixture of monomers and a suitable initiator. After the flow diverter is deployed at the aneurysm site, an ultraviolet or infrared source may be used to effect the polymerization of the monomers. The ultraviolet or infrared source can be delivered to the aneurysm site through a catheter and the source can be activated to polymerize the monomers from either the inside surface or the outside surface of the flow diverter (from the aneurysm pouch). The monomers chosen must have hydrophilic groups such that after they are polymerized, they are capable of swelling to a predetermined extent when exposed to body fluids. The swelling of the coated part of the flow diverter effectively reduces its porosity only at the region polymerized by the light source at the neck of the aneurysm.

It should be noted that the second embodiment described above may be used on any tubular device that can be placed at the location of an aneurysm. Metal devices such as those made from stainless steel, inconel, nitinol and titanium, for example, are known in the art and may be used herein with the present invention. These fibers are also used to impart radio opacity to the blood flow diverter to assist in locating the device in the patient.

As noted above, both the compression and heat set step and the in situ polymerization of a monomer on the fibers are necessary for the instant invention.

Other such devices may be made from polymers that are molded into a shape like that of the metal devices. Preferably the polymeric devices are braided from fibers, such as heat-settable polymeric fibers. Alternatively, the structure is composed of at least one heat-settable polymeric fiber co-braided with either elastomeric or non-elastomeric fibers. The fibers used can be mono-filament or multi-filament or a combination of both in order to allow for varying degrees of coverage.

### BRIEF DESCRIPTION OF THE DRAWINGS

For a more complete understanding of the invention, reference is hereby made to the drawings, in which:

FIG. 1 is a side elevational view of a blood flow diverter according to the present invention;

FIG. 2 is a side elevational view of the device of FIG. 1, with the central portion having significantly reduced porosity;

FIGS. 3a and 3b provides schematic descriptions of the coated end and a fused end of one embodiment of the blood flow diverter of this invention;

FIGS. 4a-d are schematic illustrations of the heat-set concepts of one embodiment of the present invention;

FIG. 5 is a side elevational view showing a blood flow diverter having reduced porosity in the center portion of the device as a result of polymerized coating on such portion;

FIG. 6 is a greatly enlarged portion of the device of FIG. 5 shown in the area in circle 6;

FIGS. 7a-d are a schematic description of the process of the blood flow diverter placement into a catheter and its deployment proximate an aneurysm;

### DETAILED DESCRIPTION OF THE PREFERRED EMBODIMENT

One preferred polymeric device useful as part of the present invention is produced by a sequence of steps involving the braiding of (mono- or multi-) filaments of heat-settable polymers either alone or co-braided with non heat-settable filaments of either metal or nonmetal. It is also contemplated to coat the tubular structure either partially or fully to mitigate fraying of the ends and to improve anchorage or radio opaqueness. The ends may be fused by trimming and/or heat treatment also to prevent fraying.

What is essential is that the present invention is sized to be positioned on the outside of the aneurysm rather than the prior art procedure of placing a structure inside the aneurysm. The blood flow diverter of this invention keeps blood out of the aneurysm by protecting the aneurysm from the outside. The blood flow diverter of this invention is placed over the outside of the aneurysm by a medical procedure such as by the use of a catheter and guided by a radio opaque portion. The blood flow diverter either has a decreased porosity in at least the central portion or the porosity is decreased after the blood flow diverter has been located on the outside of the aneurysm by the medical team.

If the axial compression is employed, the central portion of the device will have the desired reduced porosity, or, the braiding step can be adjusted to change the angle of the braided fibers to also decrease the porosity of the central portion. Alternatively, the fibers in the central portion can be coated with a monomer that is polymerized to increase the thickness of the fibers and reduce porosity where desired.

Additionally, another aspect of the invention is the incorporation of a number of metallic fibers by co-braiding with the polymeric fibers to provide easy detection by X-rays, i.e., to render the systems radio opaque. Radio Opacity for X-ray detection can be imparted to blood flow diverters of this invention by coating the structures using various techniques:
a) Dip coating: radio opaque powder such as tantalum is thoroughly mixed with a biocompatible adhesive to form a suspension. The ends of the braided structures or the entire structures are dipped into the suspension and the resulting coating is allowed to dry in air and then cured in an oven at 160° F. for five to ten minutes.
b) Vapor deposition: the structure to be coated and a radio opaque powder such as titanium are introduced in a vapor deposition chamber. The chamber is evacuated and then filled with an inert gas to prevent oxidation of the metal. The filaments in the chamber are then heated to a temperature high enough to vaporize the metal and deposit on the structure to be coated. Several such coatings may be required to achieve uniformity.
c) Gold sputtering: the equipment used is a sputtering machine used to coat polymers with gold for Scanning Electron Microscopy studies. The structure to be coated is introduced in the chamber filled with nitrogen/argon gas and the coating allowed to take place for several minutes, depending on the thickness desired.
d) Use of metal fibers or porous solid metal blood flow diverters by braiding a blood flow diverter from metal fibers or partially using metal fibers with other fibers such as those described above, or by casting or otherwise forming a porous blood flow diverter with less porosity in the center section.

The blood flow diverters are preferably made using braiding technology. A preferred braiding machine is a 24-carrier braiding machine. Prior to braiding the blood flow diverter, the fibers have to be wound on spindles using a winding machine. The fibers can be wound on the same spindle if there is a necessity for more than one fiber to be on the same spindle or they can be wound onto different spindles. The next step in the process is to transfer the spindles on to the braiding machine, i.e., one spindle on each carrier. The braiding process is then started and the fibers are allowed to intertwine. A mandrel of appropriate diameter and geometry is used to control the shape and size of the produced device. By changing the angle or the thickness of the fibers, the porosity can be varied as desired.

In the devices of this invention, memory can be set for a specific configuration using the heat-setting process. To achieve this, the device of this invention is shrunk or compressed together on the mandrel and secured in that configuration using tape at both ends. It is then placed in an oven and heated, such as, for example, to 150° C., in the case of polyester fiber, and then cooled to room temperature slowly. This is called heat-setting which locks-in a specific configuration in the memory of the structure. By adjusting the pressure during compression, the porosity of the center portion of the blood flow diverter can be controlled. After removing the mandrel from the oven, the blood flow diverter is taken off the mandrel and is now ready for use. In the heat-setting process, the intertwining fibers do not necessarily melt and bond together. The heat-setting process can be combined with pressure to introduce ridges at the intersection of the fibers in the blood flow diverters, and these ridges can provide additional mechanisms of memory because when the blood flow diverter is released the fibers become locked at the ridges and regain the heat-set shape.

Heat-setting can also be used to obtain various device configurations. This can be achieved by transferring the device to mandrels of the desired shapes, compressing and heat-setting as described above. Another major advantage of heat-setting is that it can be used to impart different porosities to the devices, depending on the extent of compression employed before heat-setting. The greater the compression, the lower the resulting porosity of the device. In this invention the intention is to heat set the center of the blood flow diverter to a greater compression and thus lower porosity. This forms part of the reduced porosity as does the use of a monomer polymerized in situ as described below.

The elastomeric fiber used in accordance with the invention can be natural elastomers, synthetic elastomers, or combination thereof. Natural elastomers include natural rubber. Synthetic elastomers which can be used include, but are not limited to, polyisoprene, polybutadiene and their copolymers, neoprene and nitrile rubbers, polyisobutylene, olefinic fibers such as ethylene-propylene rubbers, ethylene-propylene-diene monomer rubbers, and polyurethane elastomers, silicone rubbers, fluoroelastomers and fluorosilicone rubbers. The preferred elastomeric fibers are polyurethane or silicone elastomers. The fibers used in accordance with the present invention can be mono-filaments or multi-filament fibers and additionally they may be twisted. It should be noted that the foregoing description of the elastomeric fibers is not meant to be limiting. The elastomeric fiber of this invention can be any fiber which has sufficient elastic modulus and extensibility to provide the requisite longitudinal, torsional and radial resiliency. The preferred placement of these fibers is in the axial direction.

The non-elastomeric fibers used as a second component of the invention can be a metal fiber, a natural fiber, a synthetic fiber or any combination thereof. Examples of metal fibers that can be used include, but are not limited to, stainless steel, inconel, nitinol and titanium. Natural fibers which can be used, but are not limited to, include silk, wool and cellulosic fibers. Synthetic fibers include but are not limited to polyamides such as nylons, polyesters, rayon, polyethylene, polypropylene, polyacrylonitrile, acrylics, poly-tetrafluoroethylene, polylactic acid, polylactic/glycolic acid, and copolymers, terpolymers and derivatives thereof. The preferred non-elastomeric material is a metal, most preferred being stainless steel, inconel (a nickel/chromium/iron alloy) and nitinol. The fibers in accordance with the present invention may be monofilaments or multi-filament fibers. Additionally the fibers may be twisted. It should be noted that the foregoing description of the non-elastomeric fibers is not meant to be limiting. The non-elastomeric fiber of this invention can be any fiber which is biocompatible, corrosion resistant, and capable of providing the necessary radial rigidity, transverse elasticity, and resistance to deformation necessary for the tubular blood flow diverter.

The elastomeric fiber can be wrapped with other fibers. The wrapping can be partial or complete with respect to coverage of the surface of the elastomeric fiber with the wrapping fiber. The elastomeric fibers for this structure can be wrapped with any polymeric biocompatible fiber such as those noted above. Suitable wrapping fibers are, for example, polyesters, cotton, rayon and nylon. The preferred wrapping fiber is a polyester. The wrapping of the elastomeric fiber serves many purposes. One purpose served by the wrapping is that the elastomeric fiber is protected from damage by the other fibers in the structure (used in the braid direction). A second purpose served by wrapping is an effective means of providing the desired surface coverage to the blood flow diverter. Also, the wrapping of the elastomeric fibers helps to control and stabilize the elastic recovery of the blood flow diverter and additionally provides extra resistance in the form of friction to help keep the elastomeric fibers properly positioned. Additionally, the wrapping fibers may be treated with therapeutic agents by, growth factors, anti-coagulants, or hormones, for example, dipping the fibers into such agents. After deployment, these agents are released over time in to the body. The extent to which the elastomeric fiber is wrapped can be varied according to the desired end use and desired degree of elasticity of the material, based on the above purposes of the wrapping. Wrapping of the elastomeric fibers can be accomplished by any conventional wrapping process, including simply wrapping by hand, and, preferably, spinning fibers on to the elastomeric fiber. The elastomeric fibers can also be wrapped using continuous fibers or filaments, or discrete fibers, or staple fibers, such as cotton. The diameter of the wrapping fibers can be of nano or micro-denier.

The first component and the second component can be intertwined by any method, but are preferably braided in a conventional manner. Methods of braiding fibers and braiding in general have been described in literature; see, e.g., Ko, Frank K, “Braiding”, Manufacturing Processes, (1987) and Ko, Frank K., “Preform Fiber Architecture for Ceramic-Matrix Composites”, Ceramic Bulletin, Vol. 68, No. 2, (1989), the entire contents of which are herein incorporated by reference.

In one embodiment, the ends of the blood flow diverter are preferably coated to prevent fraying. By coating the ends of the blood flow diverter and preventing fraying, damage to the blood vessel walls and difficulty in handling can be minimized. Also, by providing the coatings on each end of the blood flow diverter with some extra texture, anchorage of the prosthesis within the blood vessel can be improved. Suitable coatings may include any biocompatible material. The coating should also preferably be an elastomeric material, such as biocompatable silicones or polyurethanes. The coating can be applied by dipping the end of the blood flow diverter into a curable silicone or polyurethane coating composition, and subsequently curing the coating. Heat or ultra-violet light can be applied in some cases to enhance the cure rate. Brushing or spraying of the coatings can also accomplish coatings of the ends of the prosthesis. Curing of the coatings can be accomplished by any conventional method, however, self-curing and ambient-curable coatings are preferred as heat can damage the elasticity of the elastomeric materials in the prosthesis. Additionally, self-curing and ambient-curable coatings shrink less upon cure and are also preferable for that reason. Generally, the coating should be applied to the extent necessary to reduce fraying, which in most cases is about four picks or intersections of the intertwined fibers. This can be accomplished by coating from about 1 mm to about five mm as measured longitudinally on each end of the tubular prosthesis. However, the coating may extend further if desired and, indeed, if the intended application permits, can cover the entire prosthesis. Generally, tubular prostheses, which require less porosity, can permit use of more extensive coatings, and tubular prostheses, which may require substantial cutting of length, may also have more extensive end coatings. The thickness of the coating can be about from 1 micron to about 500 microns, and is preferably from about 20 microns to about 200 microns.

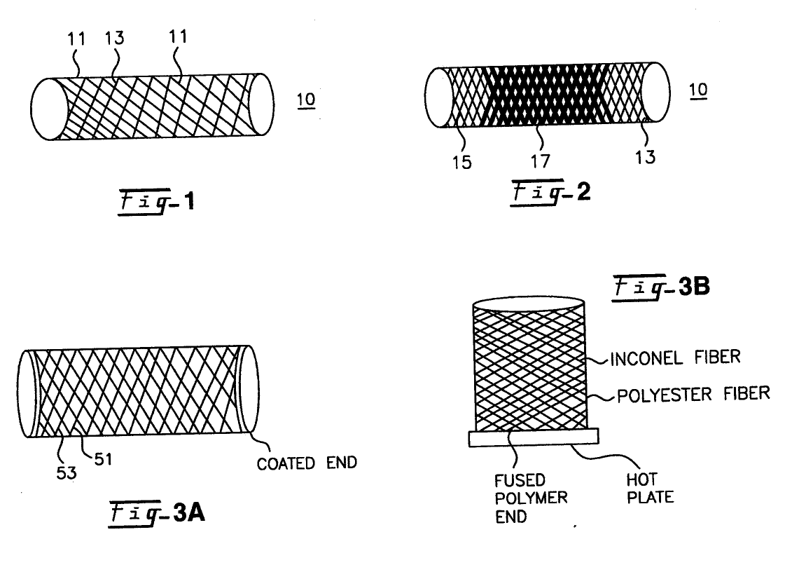

Turning now to the drawings, FIG. 1 shows a device 10, generally, for use as vascular blood flow diverter in accordance with the invention. The tube 11 is made from heat-settable and non-elastomeric fibers 13 are disposed in an helical configuration, wherein oppositely wound helical fibers 15 are intertwined over and under each other. The elastomeric fibers in the center, at this point, are the same thickness as fibers 11 and 13.

FIG. 2 shows a view of the same tube 11 in which the space between the fibers 17 in the center of the tube 11 are closer together, either by changing the angle of braiding or by compressing the center portion.

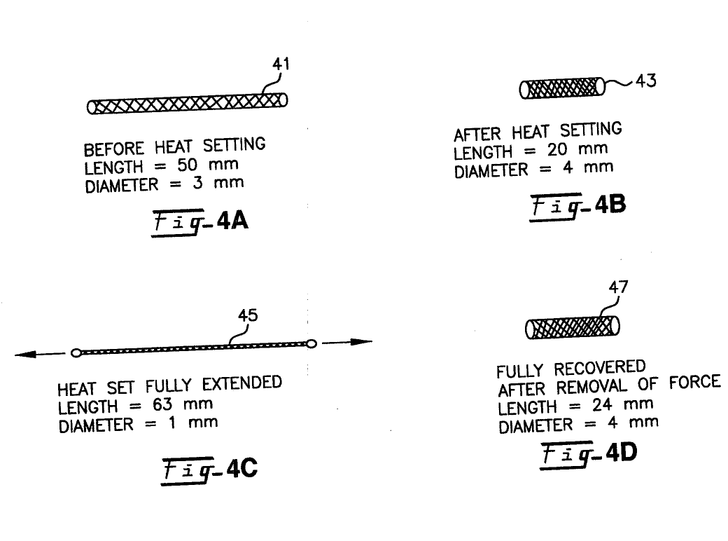

FIGS. 3A and 3B shows a slightly enlarged tube illustrating the technique of heat sealing the ends of the tubular blood flow diverter to prevent sharp fiber ends from harming the patient.

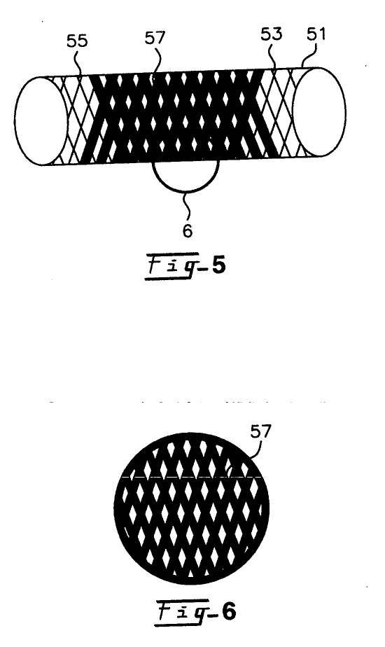

FIGS. 4A, 4B, 4C and 4D represent the preferred embodiment of the present invention, where a braided blood flow diverter 41, is formed, as described above, so that the blood flow diverter has a predetermined length. All that is required is that the blood flow diverter 41 have at least some fiber which is capable of holding a heat-set when applied as described herein. Typical dimensions might be a length of 50 mm and a diameter of 3 mm. Preferred examples of heat-set capable fibers have been listed above. In FIG. 4B, the blood flow diverter 43 has been compressed axially to a shorter length and subjected to heat treatment to heat-set blood flow diverter 43 in this embodiment, so that the length, for example only, might be 20 mm and the diameter 4 mm, since the same amount of fiber is present. This heat-set version of the blood flow diverter 43 may be coated, as described below, or used as is. When blood flow diverter 43 is used, it is extended as shown in FIG. 4C as blood flow diverter 45, having an extended length of, for example, 63 mm, which results in a small diameter of 1 mm, for insertion into a body vessel as intended. When the force of extending the blood flow diverter 75 is released after placement in the body, the blood flow diverter 47 in FIG. 4D recovers to a useful size, such as, for example, one with a length of 24 mm and a diameter of 4 mm. It is to be understood that this compression and heat set is one part of the two part porosity reduction of the present invention.

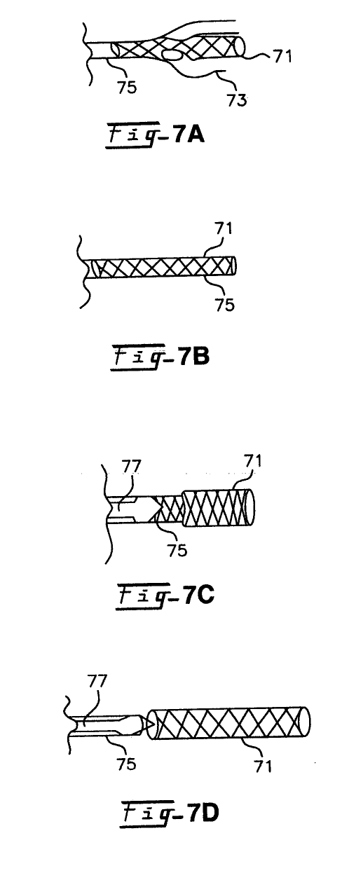

FIG. 5 shows a preferred structure 51 in accordance with the invention wherein fibers 53 and 55 are of one thickness and center portion fibers 57 are much thicker as a result of compression and heat set as in FIGS. 4A-D and with the addition of a polymerized coating giving a further decrease of the porosity at that center portion of blood flow diverter 51. FIG. 6 shows the enlarged fibers 57 both as compressed and coated.

The incorporation of non-elastomeric metal fibers is primarily for the purpose of radio opacity and in certain cases does provide the additional feature of helping in the blood flow diverter anchorage. The preferred non-elastomeric fibers are metallic fibers. Non-metallic, non heat-settable polymeric fibers may also be used in conjunction with heat-settable fibers as described above in order to tailor the physical and mechanical properties of the blood flow diverters. In such cases, these fibers can be any of the single or multi-filament elastomeric or non-elastomeric fibers described above.

Blood flow diverters with high coverage can be made with 9 mono-filament fibers, 9 multi-filament fibers of the same material and 6 metal fibers, each mounted on separate carriers and then braided into a blood flow diverter. The blood flow diverter can be braided at a low angle to have a high surface coverage or can be brained at a high angle, then reduced in length on the mandrel and heat-set. Wrapping significantly enhances surface coverage. Blood flow diverters with very high coverage can be made with 18 mono-filament and 18 multi-filament fibers, one of each type wound on the same spindle and braided along with six metal fibers. Blood flow diverters requiring low thickness can be made with 12 mono-filament fibers, 6 multi-filament fibers and 6 metal fibers as above. Another example is 12 mono-filament fibers, 6 of them co-wound with 6 multi-filament fibers only and 6 metal fibers.

Blood flow diverters made with only 18 mono-filament fibers and 6 metal fibers each type on separate spindles or one metal and one mono-filament co-braided onto the same spindle are also contemplated.

FIGS. 7A, 7B, 7C and 7D are schematic drawings showing the preferred process of this invention as presently contemplated, for blood flow diverter placement into a catheter and its deployment proximate an aneurysm. The blood flow diverter 71 is squeezed by hand 73 and manually inserted into the catheter 75 as shown in FIG. 7A. Once the blood flow diverter is lodged completely inside the catheter 75 as in FIG. 7B, the catheter is introduced into the body at the aneurysm using standard techniques used currently by surgeons in blood flow diverter deployment and pushed to the location where it needs to be deployed or placed. At the desired location proximate the aneurysm, the blood flow diverter is pushed out of the catheter using another catheter, shown in FIGS. 7C and 7D, typically a balloon catheter 77 in the deflated state.

It will be appreciated by those skilled in the art that changes could be made to the embodiments described above without departing from the broad inventive concept thereof. While particular embodiments of the present invention have been illustrated and described, it is not intended to limit the invention to any specific embodiment. The description of the invention is not intended to limit the invention, except as defined by the following claims.

1. A blood flow diverter device for treatment of intracranial aneurysms, comprising:
a porous tubular member formed from braided thermoplastic fibers having a central portion and two ends, said member being of sufficient flexibility and body compatibility to be placed in proximity with an aneurysm;
said braided fibers being compressed to decrease the porosity of said central portion and heat set to preserve said decrease; and
said central portion of said tubular member having further decreased porosity by coating said central portion with a monomeric coating in an amount sufficient to decrease the porosity of said central portion upon polymerization of said monomeric coating; and including a polymerization initiator for polymerizing said monomeric coating upon command to cause said decreased porosity to block said blood flow after said device has been placed in proximity with an aneurysm;
whereby said central portion has reduced porosity to block blood flow from entering through the aneurysm.

2. The device of claim 1, wherein said two ends of said tubular member have sufficient porosity to keep open small perforator arteries proximate said intracranial aneurysm.

3. The device of claim 1, wherein said monomers contain reactive double bonds capable of undergoing polymerization triggered by an ultraviolet or infrared source.

4. The device of claim 3, wherein said monomer is selected from the group consisting of acrylate or methacrylate monomer with pendant hydrophilic groups such as acrylic or methacrylic acid and their salts; hyroxyalkyl acrylate or methacrylate; and ethoxyalkyl acrylate or methacrylate; acrylamide and derivatives; vinyl pyrollidone and derivatives; vinyl pyridine and derivatives; styrene sulfonate and vinyl monomers containing quaternary ammonium salts; and mixtures thereof.

5. The device of claim 3, which further includes at least one hydrophobic monomer in an amount sufficient to limit the degree of swelling of said monomeric coating after polymerization of said monomeric coating.

6. The device of claim 5, wherein said hydrophobic monomer is selected from the group consisting of styrene, methyl acrylate or methacrylate, phenylethyl acrylate or methacrylate and mixtures thereon.

7. The device of claim 1, wherein said monomeric coating further includes a cross-linking monomer for controlling swelling and preventing the resulting polymer from dissolving, said cross-linking monomer having two or more active double bonds per molecule.

8. The device of claim 7, wherein said cross-linking monomers is selected from the group consisting of divinyl benzene; allyl acrylate or methacrylate; di- or tri-acrylates or methacrylates; di-or tri-alkoxyacrylates or alkoxymethacrylates and mixtures thereof.

9. The device of claim 1, which further includes a metallic fiber positioned to give radio opacity to said blood flow diverter.

10. The device of claim 1, wherein braided fibers are braided at an angle with respect to the axis of said tubular member in said central portion to decrease the porosity of said central portion, and then compressed and heat set to form an expandable device upon insertion into the body.

11-20. (canceled)
